(19) United States
(12) Patent Application Publication
(10) Pub. No.: US 2008/0031819 A1
(43) Pub. Date: Feb. 7, 2008

(54) Compositions and Methods that Enhance Articular Cartilage Repair

(75) Inventors: Ernest F. Terwilliger, Boston, MA (US); Magali Cucchiarini, Homburg (DE)

Correspondence Address: CLARK & ELBING LLP, 101 FEDERAL STREET, BOSTON, MA, 02110, US

(73) Assignee: Beth Israel Deaconess Medical Center, Boston, MA (US)

(21) Appl. No.: 11/628,810

(22) PCT Filed: Jun. 9, 2005
(86) PCT No.: PCT/US05/20278

§ 371 Date: Jul. 31, 2007

Related U.S. Application Data
(60) Provisional application No. 60/578,177, filed on Jun. 9, 2004.

(51) Int. Cl. A61K 48/00; A61K 49/00; A61P 43/00; C12N 15/00

(52) U.S. Cl. 424/9.2; 435/320.1; 514/44

(57) ABSTRACT

In general, this invention relates to compositions and methods useful in enhancing articular cartilage repair by providing for the sustained release of growth factors to articular cartilage cells to induce cell proliferation and extracellular matrix synthesis.

### STATEMENT AS TO FEDERALLY SPONSORED RESEARCH

The present research was supported by a grant from the National Institutes of Health/National Cancer Institute (Number R21AR48413). The U.S. government has certain rights to this invention.

### BACKGROUND OF THE INVENTION

In general, this invention relates to compositions and methods useful in enhancing articular cartilage repair by providing for the sustained release of growth factors to articular cartilage cells to induce cell proliferation and extracellular matrix synthesis.

Articular cartilage is a tough, elastic tissue that covers the ends of bones in joints and enables the bones to move smoothly over one another. Articular cartilage damage may result from acute trauma or from osteoarthritis. Osteoarthritis, which afflicts 32 million Americans, is a leading cause of disability in the United States. When articular cartilage is damaged, it does not heal as rapidly or effectively as other tissues in the body. In fact, natural healing of damaged cartilage in adults is poor to negligible because adult chondrocytes have a limited capacity for proliferation and new matrix synthesis.

Injured adult hyaline articular cartilage does not heal effectively, and defects either remain empty or become filled with functionally inferior fibrous tissue. Current therapeutic options are available for hyaline articular cartilage lesions are diverse, yet none can predictably restore integrity and function. Similarly, treatments for osteoarthritis are primarily intended to alleviate its symptoms, rather than reverse the underlying cartilage erosion.

In recent years, growth factors and other agents that regulate cartilage homeostasis and augment cartilage reparative activity have been identified. Growth factors could improve the healing of osteochondral cartilage defects by stimulating the proliferation of cells that fill the defect and increasing their synthesis of extracellular matrix proteins. These beneficial effects are, however, impeded by the short pharmacological half-lives of growth factors. Direct articular injection of a growth factor results in its clearance within a few minutes and cartilage healing in response to growth factor delivery is incomplete. Systemic delivery has the additional complication of unwanted side-effects.

There exists a need for improved therapeutics for articular cartilage repair and, in particular, therapeutics that can induce articular cartilage cells to undergo proliferation and extracellular matrix synthesis.

### SUMMARY OF THE INVENTION

We have discovered methods and compositions for effectively inducing articular cartilage repair.

In one aspect, the invention generally features a method for enhancing cartilage repair in a subject, the method includes administering to the subject having cartilage damage at least one vector (e.g., AAV-1, AAV-2, AAV-3, AAV-4, AAV-5, AAV-6, adenovirus) encoding a therapeutic polypeptide, or fragment thereof, selected from the group consisting of FGF-2, IGF-1, and IGF-1 receptor. Preferably, a viral vector encoding a therapeutic peptide is administered directly by injection to a region of articular cartilage damage. This direct administration results in the transduction of cells in the region, which subsequently stably express a therapeutic peptide. In one preferred embodiment, the vector is AAV-2. In another preferred embodiment, the therapeutic polypeptide is FGF-2. In another preferred embodiment, at least two or three vectors encoding at least two or three therapeutic polypeptides are administered. In one preferred embodiment, one vector encodes an IGF-1 polypeptide and a second vector encodes an IGF-1 receptor polypeptide. In another preferred embodiment, the first vector encodes an FGF-2 polypeptide and the second vector encodes an IGF-1 polypeptide. In some embodiments, the cartilage damage results from trauma or osteoarthritis. In another embodiment, the vector is administered to a joint selected from the group consisting of knee, ankle, foot, hip, spine, wrist, elbow, and shoulder.

In a related aspect, the invention features an AAV vector comprising an open reading frame that encodes an IGF-1 or IGF-1 receptor polypeptide, or a fragment thereof. In one embodiment, the vector further contains an open reading frame that encodes an FGF-2 polypeptide, or a fragment thereof. In another embodiment, the vector contains a promoter operably linked to the nucleic acid molecule that is capable of driving the expression of the nucleic acid molecule in a specific cell type, tissue, or organ.

In another related aspect, the invention features a cell containing the vector of the previous aspect.

In another aspect, the invention features a cartilaginous cell comprising an AAV vector that encodes FGF-2, or a fragment thereof.

In another aspect, the invention features a method for identifying a candidate polypeptide that enhances cartilage repair. The method involves contacting an organism with cartilage damage with at least one vector (e.g., AAV-1, AAV-2, AAV-3, AAV-4, AAV-5, AAV-6, or adenovirus) that encodes a candidate polypeptide (e.g., a growth factor or a growth factor receptor polypeptide); and (c) detecting cartilage repair in the organism relative to a control organism not contacted with the vector, where the repair indicates that the candidate polypeptide enhances cartilage repair. In one preferred embodiment, the vector is administered directly to an articular joint.

In another aspect, the invention features a pharmaceutical composition comprising an AAV vector that encodes an IGF-1 polypeptide or an IGF-1 receptor polypeptide and an excipient.

In another aspect, the invention features a kit comprising an AAV vector that encodes FGF-2, IGF-1, or an IGF-1 receptor and instructions for administering at least one of the vectors to a subject having articular cartilage damage.

By “articular cartilage” is meant any cartilage that covers the articular surface of a bone.

By “enhances articular cartilage repair” is meant facilitates cell proliferation or extracellular matrix synthesis in a joint or promotes healing of damage.

By “fragment” is meant a portion of a polypeptide or nucleic acid that is substantially identical to a reference protein or nucleic acid, and retains at least 50% or 75%, more preferably 80%, 90%, or 95%, or even 99% of the biological activity of the reference protein or nucleic acid.

By “FGF-2” is meant a basic fibroblast growth factor polypeptide, or fragment thereof, that stimulates chondrocyte proliferation or articular cartilage repair. An exemplary FGF-2 polypeptide is described by Seno et al. (Cytokine 10:290-4, 1998). Other exemplary FGF-2 polypeptides include NP_{—}001997 and the polypeptide encoded by NM_{—}002006.

By “IGF-1” is meant an insulin-like growth factor I polypeptide or fragment thereof, that stimulates chondrocyte proliferation, extracellular matrix synthesis, or articular cartilage repair. Exemplary IGF-1 polypeptides include GenBank Accession Nos. X00173, CAA40093, CAA40092, the IGF-1 polypeptides encoded by X56774 and X56773, and those polypeptides described by Jansen et al. (Nature 306:609-11, 1983).

By “IGF-1R” is meant an insulin-like growth factor I polypeptide receptor that binds IGF-1 and stimulates articular cartilage repair or chondrocyte proliferation. An exemplary IGF-1R is described by Pedrini et al. (Biochem Biophys Res Commun. 202:1038-46, 1994). Other IGF-1 receptors include NP_{—}000866 and the IGF-1R encoded by NM_{—}000875.

By “joint” is meant a point of articulation between two or more bones (e.g., knee, elbow, hip, shoulder, wrist, spinal joints, hand, finger, wrist joints, and feet).

By “positioned for expression” is meant that the polynucleotide of the invention (e.g., a DNA molecule) is positioned adjacent to a DNA sequence which directs transcription and translation of the sequence (i.e., facilitates the production of, for example, a recombinant polypeptide of the invention).

By “transformed cell” is meant a cell into which (or into an ancestor of which) has been introduced, by means of recombinant DNA techniques, a polynucleotide molecule encoding a polypeptide.

By “transgene” is meant any piece of DNA that is inserted by artifice into a cell. Such a transgene may include a gene that is partly or entirely heterologous (i.e., foreign) or may represent a gene homologous to an endogenous gene of the organism.

By “therapeutic vector” is meant a vector that encodes a polypeptide that affects the function of an organism. A therapeutic vector may decrease, suppress, attenuate, diminish, arrest, or stabilize the development or progression of a disease or disorder in a subject.

The invention features methods and compositions for enhancing articular cartilage repair. These compositions and methods facilitate cell proliferation and extracellular matrix synthesis in joints having articular cartilage damage. Other features and advantages of the invention will be apparent from the detailed description, and from the claims.

### BRIEF DESCRIPTION OF THE DRAWINGS

FIGS. 1A and 1B are micrographs showing hemaglutinin (HA) tag reactivity in rabbit distal femurs transduced with a negative control vector (FIG. 1A) (AAV-RFP) or with a recombinant adeno associated virus vector encoding IGF-1, which is fused to a hemaglutinin tag (FIG. 1B) (AAV-HA-IGF-1).

FIGS. 2A and 2B are micrographs showing red fluorescent protein expression in rabbit distal femur cells 21 days after the cells were transduced with either AAV-RFP (FIG. 2A) or with AAV-HA-IGF-1 (FIG. 2B).

FIGS. 3A and 3B are photomicrographs of femoral tissue sections stained for proteoglycan with Safranin O twenty-one days following transduction with AAV-RFP (FIG. 3A) or with AAV-HA-IGF-1 (FIG. 3B).

FIGS. 4A and 4B are photomicrographs showing β-galactosidase (β-gal) reactivity in rabbit full-thickness defects four months after transduction with either an AAV vector encoding FGF-2 (Panel A) or an AAV vector encoding β-galactosidase (AAV-lacZ). The primary antibody was mouse anti-βgal (GAL-13; Sigma): 1:50 dilution overnight at 4° C.

FIGS. 5A and 5B are photomicrographs showing human FGF-2 immunoreactivity four months after transduction with either a control AAV vector encoding β-galactosidase (β-gal) (Panel A) or an AAV vector encoding FGF-2 (Panel B).

FIGS. 6A and 6B are photomicrographs showing Safranin O staining for proteoglycan in full-thickness defects four months after transduction with either a control AAV vector encoding β-galactosidase (AAV-lacZ) (FIG. 6A) or an AAV vector encoding FGF-2 (FIG. 6B).

FIGS. 7A and 7B are photomicrographs showing collagen type II staining immunoreactivity in full-thickness defects four months after transduction with an AAV vector encoding β-galactosidase (AAV-lacZ) (FIG. 7A) or an AAV vector encoding FGF-2 (FIG. 7B).

FIG. 8 is a schematic diagram of AAV vectors described herein. Abbreviations: AAV (Adeno Associated Virus); RFP (Red Fluorescent Protein); SVpA (SV40 small t intron and polyadenylation signal); ITR (Iterated Terminal Repeat). IGF-1R (IGF-1 receptor).

### DETAILED DESCRIPTION OF THE INVENTION

The present invention provides compositions and methods for delivering growth factors to joints to facilitate articular cartilage repair.

Using a rabbit model of articular cartilage injury, we discovered that (i) adeno associated virus (AAV) vectors can be used to transduce articular cartilage cells; (ii) these vectors can be used for the long-term delivery of growth factors; and (iii) FGF-2 is surprisingly effective at facilitating the healing of introduced defects in articular joints in vivo, promoting chondrocyte proliferation, accompanied by enhanced filling of the defects and improved global architecture.

Recombinant Therapeutic AAV Vectors

AAV is a non-pathogenic, replication-defective parvovirus that exhibits a number of characteristics that enable it to efficiently and persistently transduce cells present in joints. Because it is one of the smallest human DNA viruses, just about 25 nm in diameter, it penetrates the extracellular matrix more easily than other classes of vectors. In addition, AAV exhibits minimal cellular immunogenicity, in part because standard AAV vectors carry no viral coding sequences. In addition, the simplicity of the viral capsid, which is composed of several variations of a single major polypeptide generated by alternate splicing events, also contributes to the low immunogenicity of AAV. Because AAV is a poor adjuvant, as well as a poor immunogen, its use in heterologous transgene expression is less likely to induce a destructive host immune response. This is particularly true for the vector's use in immunologically sequestered sites, such as joints (Fisher et al., Nat Med 3:306, 1996; Xiao et al., J. Virol. 70: 5098-8108, 1996).

AAV is capable of delivering transgene cassettes that are up to 5 kilobases in length. While wild-type AAV integrates in a specific chromosome region, recombinant AAV integrates slowly and non-specifically. This characteristic allows the vector to persist as an episome that remains stable for months or even years in non-dividing cells allowing for the sustained delivery of healing agents.

AAV-2 therapeutic vectors were produced using pSSV9, a modified genomic clone of AAV-2 (Madry et al., Hum Gene Ther 14: 393-402, 2003). Our standard AAV vector plasmid derived from pSSV9, pACP, contained a promoter element (immediate early promoter of cytomegalovirus (CMV-IE)), a multiple cloning site for gene inserts, an SV40 small t intron, and a polyA signal. AAV inverted terminal repeats (ITRs), which are required for viral packaging, flank these regions.

The gene inserts present in the AAV vectors included a modified IGF-1 (Jansen et al., Nature 306: 609-11, 1983), IGF-I receptor (IGF-IR) (Pedrini et al., Biochem Biophys Res Commun. 202:1038-46, 1994), and FGF-2 (Seno et al., Cytokine 10: 290-4, 1998). The AAV-2 therapeutic vectors do not include native AAV gene coding sequences. We note that the cDNA for the IGF-1 receptor, the longest of the protein gene sequences, is still within the 5 kb packaging limit of the vectors. A 4.0 Kb fragment containing the human IGF-1 receptor cDNA was cloned into the unique Xba I site inserted in the standard AAV-2 vector plasmid, pACP. Similarly, a 0.48 Kb fragment containing the human FGF-2 cDNA was cloned between the Xba I and Sal I sites in pACP and a 0.54 Kb fragment containing a modified human IGF1 cDNA was cloned between the Xba I and Hind III sites in pACP.

Other AAV vectors constructed included vectors expressing either β-galactosidase (Beta-gal) (Du et al., Gene Therapy 3:254-261, 1996) or red fluorescent protein (CLONTECH INC Franklin Lakes, N.J.), which is derived from a species of coral. We have previously constructed and successfully used AAV expressing Green Fluorescent Protein (GFP) (Inouye et al., J. Virology. 71:4071-407, 1997). Red Fluorescent Protein exhibits less autofluorescence in most tissues at the longer wavelengths where this marker emits (peak emission 583 nm). The fluorescent markers also have the advantage of being detectable in living cells, by virtue of their innate fluorescence, as well as in fixed cells and tissues by immunocytochemistry using commercial antibodies.

AAV Vectors Transduced Cartilage Cells In Vitro and In Vivo

AAV was used to transduce cells of cultured cartilage discs (Madry et al., Trans Orthop Res Soc 46: 305, 2000) and rabbit knees in vivo, as described below. Neonatal bovine, or normal or osteoarthritic human articular cartilage explant cultures were directly transduced with the AAV-lacZ vector. This transduction resulted in long-term lacZ gene expression in each explant. This expression was maintained until 150 days post-transduction. Persistent and efficient gene transfer was also carried out in normal or osteoarthritic articular human chondrocytes in culture and in neonatal bovine chondrocytes in culture.

AAV Delivered FGF-2 and IGF-1 to Chondrocytes In Vitro

For vectors encoding therapeutic factors, expression of each factor was confirmed in vitro. FGF-2 expression and secretion was confirmed in cultured human chondrocytes by enzyme-linked immunosorbent assay (ELISA) (R&D SYSTEMS, Minneapolis, Minn.). 0.1 million chondrocytes/well of a 12-well plate were transduced with 40 ul of AAV-FGF-2. Cells were exposed to the vector for 90 minutes in a minimal amount of serum free medium, after which serum-containing medium was added back and the cells were incubated in the residual vector overnight. Four days after transduction, 150 pg/ml FGF-2 was detected in the culture medium using an enzyme-linked immunosorbent assay (ELISA) (R&D Systems, Minneapolis, Minn., USA). Over 150 pg/ml FGF-2 could routinely be detected in the culture medium following transduction. The assay background averaged less than 8 pg/ml. This protein was determined to be biologically active in a proliferation assay on fresh chondrocytes using supernatant media from cells transduced with the AAV-FGF-2 vector.

A modified IGF-1 cDNA transgene was inserted in the standard AAV-2 vector plasmid, pACP. In this transgene, an efficient leader sequence and secretion signal, derived from the V-J2-C region of the mouse immunoglobulin kappa chain, was followed by an HA tag and then by the coding sequence of the full length IGF-1 pre-protein, including the 35 amino acid C-terminal peptide (Jansen et al., Nature 306: 609-11, 1983). This 0.54 Kb fragment was cloned between the Xba I and Hind III sites in pACP. This vector yielded the highest amounts of secreted IGF among a set of related constructs. IGF-1 production was tested by ELISA of culture media. Transduction of chondrocytes with 40 ul of the new IGF-I vector yielded in the range of 3.6-5.0 ng ml of IGF-I in human chondrocyte supernatant medium after 4 days. The biological activity of the protein was confirmed in proliferation assays on fresh chondrocytes using supernatant media from cells transduced with the vector.

AAV Vectors Expressed Proteins in Rat Knee Joints In Vivo

As described below, AAV therapeutic vectors were also used to transduce articular cartilage cells in knee joints in vivo. Osteochondral defects, 1 mm diameter, were produced in the femoral articular surface of the femoropatellar joint of female Sprague-Dawley rats, using a drill and a partial thickness chondral defect (2 mm²) was created in the medial femoral condyle using a scraper. An AAV-lacZ vector was applied directly to these defects. Three or ten days after this application, histological analysis of serial sections revealed intense X-gal staining in the defects. The X-gal staining was predominantly present in cells of the repair tissue that filled the osteochondral defects, and in chondrocytes surrounding the defects. X-gal staining was also present in parts of the synovium. This staining was not observed in mock-transfected knee joints. Similar findings were obtained with an AAV-RFP vector (Madry et al., Hum Gene Ther 14: 393-402, 2003).

AAV Vectors Expressed Proteins in Rat Knee Joints In Vivo

Osteochondral defects were introduced in the femoral articular surface of the femoropatellar joints of Chinchilla bastard rabbits (mean weight: 2.8±0.4 kg). A 3.2 mm full-thickness osteochondral defect was created in the patellar groove of each knee. After washing the defects with phosphate buffered saline, 10 ul of an AAV was applied to each defect. Each animal received an AAV encoding AAV-FGF-2, AAV-IGF-1R, or AAV-IGF-1 on one knee, and AAV-lacZ or AAV-RFP on the other knee. Treatments were evenly distributed between right and left knees.

Rabbits were euthanized at various times following vector administration, and the distal femoras with adjacent synovium were removed. For time points at 3 days, 10 days, and 3 weeks, each treatment group contained 2-3 animals. For later time points, each treatment group contained 6-7 animals.

Retrieved distal femora were fixed in 10% formalin and decalcified for 4 weeks in 10% Na3C6H5O7.2H2O, 22.5% formic acid. Paraffin-embedded sections (5 um) were prepared using a paraffin station (Leica EG 1140C) and a manual microtome (Leica RM 2135). The sections were then treated to detect the expressed protein as described below.

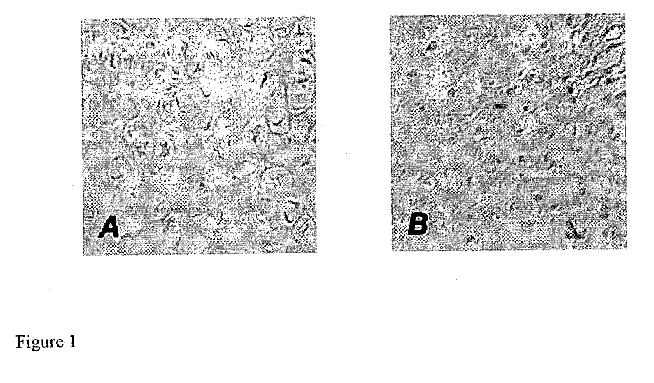

For IGF-1 HA, a primary antibody against the HA peptide tag (mouse anti-HA (H9658; Sigma) was used at 1:1,000 dilution, overnight at 4° C. Sections were then treated with a 1:200 dilution of goat anti-mouse biotinylated antibody (VECTOR LABORATORIES, Burlingame, Calif.) for 1 hour at room temperature and the VECTASTAIN kit (VECTOR LABORATORIES, Burlingame, Calif.) was used to visualize immunoreactivity. Results are shown in FIGS. 1A and 1B. Stained sections were examined under a bright-field microscope and IGF-1 protein expression was observed (FIG. 1B). Staining with an anti-IGF-1 antibody yielded similar results.

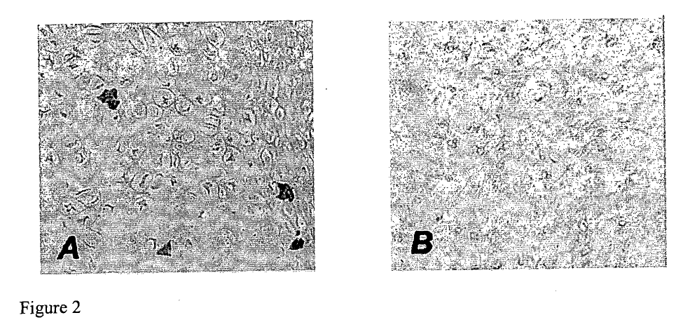

Control knees receiving only AAV-RFP exhibited strong immunoreactivity for RFP for at least 3 weeks after vector administration. RFP expression was detected using a monospecific antibody, rabbit anti-RFP (DsRed; CLONTECH BD BIOSCIENCES, Franklin Lakes, N.J.), at 1:1,000 dilution overnight at 4° C. The rest of the development procedure was carried out as described above. Results are shown in FIGS. 2A and 2B.

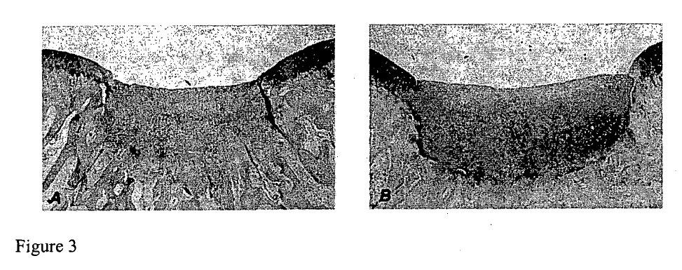

Comparisons of Safranin O staining, as well as immunoreactivity against Type I and Type II collagen, revealed no differences between the untreated and AAV-FGF-2 treated knee groups at Day 10. By Day 20, healing of the defects had begun, and levels of Type II collagen were higher in knees injected with either rAV-FGF-2 or AAV-IGF-1R vectors relative to control knees. Sections from this time point were stained with 0.02 percent Fast Green (5 minutes), then washed with 1 percent acetic acid (3×30 minutes) and visualized with 1 percent Safranin O (30 minutes) to detect proteoglycans. Results are shown in FIGS. 3A and 3B. Safranin O staining of sections from this early time point revealed zones of intense proteoglycan staining in the treated defect receiving AAV-IGF-1 (FIG. 3B). This intense staining was not observed in knees receiving only AAV-RFP (FIG. 3A).

Protein expression was assayed at four months, in rabbits that received AAV-FGF, AAV-IGF1R, or a control vector. Unlike the short time points, where each group comprised 2-3 animals, each treatment group at this longer time point consisted of 6-7 animals.

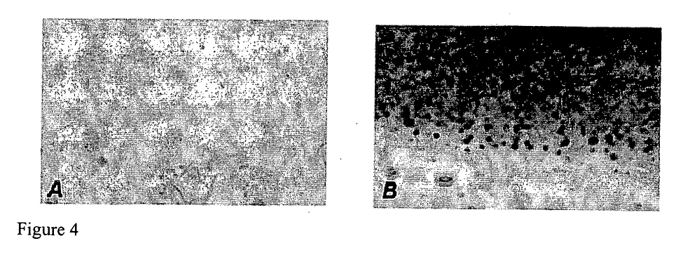

Beta-galactosidase (Beta-gal) activity is still readily detected after 4 months using an anti-Beta-gal antibody (MAB 1802, CHEMICON INTERNATIONAL, Temecula, Calif.) in control knee joints that received only AAV-lacZ. Immunoreactivity is still present in many of the cells that form the repair tissue within and surrounding the defects as well as in synovial and muscle cells of parts of the quadriceps muscle adjacent to the patella and in the infrapatellar fat pad as well as in the extracellular matrix. The marrow-derived cells that fill the defect are prominent among the cells still expressing beta-Gal (FIGS. 4A and 4B).

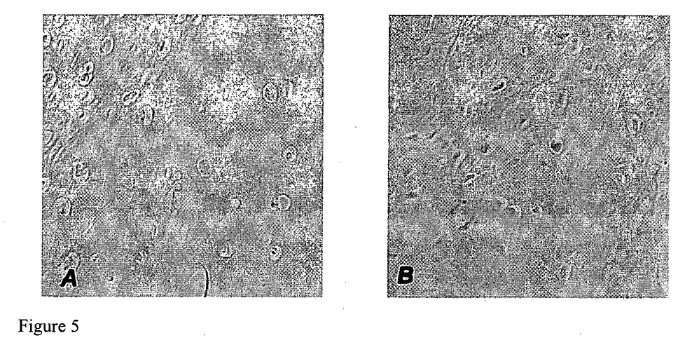

FGF-2 was also detected by immunohistochemistry at the 4 month time point in knees injected with the AAV-FGF-2 vector (FIGS. 5A and 5B), although the intensity of the staining was somewhat reduced relative to the staining observed at earlier time points. The primary antibody was specific for the human form of the protein and had no crossreactivity with corresponding rabbit proteins (Ab-3, ONCOGENE RESEARCH PRODUCTS, San Diego, Calif.). For these experiments, the tissues were prepared as described above. The primary antibody used was mouse anti-FGF-2 (GF22 or Ab-3; ONCOGENE RESEARCH PRODUCTS, San Diego, Calif.) at 1:100 dilution, incubated overnight at 4° C.

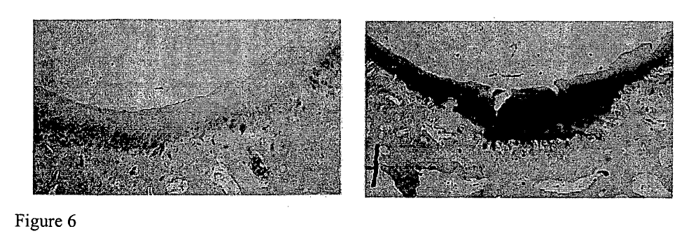

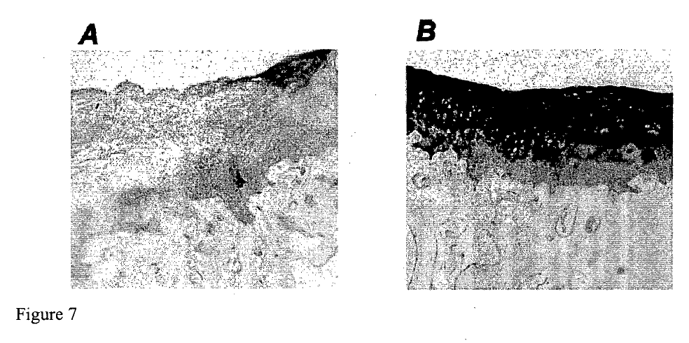

To assess repair at 4 months, sample sections of treated as well as control knees were stained with Safranin 0, which stains proteoglycans as an index of extracellular matrix synthesis (FIGS. 6A and 6B). Sections were stained with 0.02 percent Fast Green (5 minutes), then washed with 1 percent acetic acid (3×30 min) and visualized with 1 percent Safranin O (30 minutes). Defect repair was further characterized using specific antibodies against collagen Type II (FIGS. 7A and 7B), a major component of mature hyaline cartilage. Comparisons of Safranin O staining, as well as reactivities against Type I and Type II collagen, revealed no differences between the untreated and AAV-FGF-2 treated knee groups at Day 10. At Day 20, some healing had started, and levels of Type II collagen appeared higher in knees injected with either AAV-FGF-2 or AAV-IGF-1R than in control knees. Immunocytochemistry was carried out using a mouse anti-collagen II primary antibody (AF-5710; DPC—Acris) at 1:50 dilution, overnight at 4° C. Antibody reactivity was visualized using a goat anti-mouse biotinylated IgG (VECTOR LABORATORIES, Burlingame, Calif.) 1:100 dilution for one hour at room temperature, DAB, and the VECTASTAIN (VECTOR LABORATORIES, Burlingame, Calif.) kit.

These results were even more dramatic 4 months after transduction, when florid proteoglycan staining lined the defects in knees treated with AAV-FGF-2 and the defects were largely filled in. The defects in treated knees also exhibited staining for collagen II that was more regular as well as consistent with staining seen in surrounding healthy cartilage. In marked contrast to the AAV-FGF-2 treated knees, relatively little healing had occurred in the control knees. Sections were stained with 0.02 percent Fast Green (5 min), then washed with 1 percent acetic acid (3×30 min) and visualized with 1 percent Safranin O (30 minutes).

To quantify the results at four months, for each knee, at least 10 Safranin O sections were analyzed for 8 different parameters, including extent of filling of the defect, integration of repair tissue with the surrounding cartilage, cellular morphology (rounded chondrocyte morphology versus spindle shaped fibroblast morphology) and defect architecture (voids, clefts, or fibrillations). (See Table 1)

| | Category | | | | | | | | | | | | | | | | | |
|---|---|---|---|---|---|---|---|---|---|---|---|---|---|---|---|---|---|---|
| | Scored by | | Filling of | | | | Matrix | | Cellular | | Architecture | | Architecture | | New subch. | | | |
| | Individual | | the defect | | Integration | | staining | | morphology | | (defect) | | (surface) | | bone | | Tidemark | |
| | Rabbit: | Slide # | #1 | #2 | #1 | #2 | #1 | #2 | #1 | #2 | #1 | #2 | #1 | #2 | #1 | #2 | #1 | #2 |
| FGF-2 | | | | | | | | | | | | | | | | | | |
| | K 054 | A | 0 | 0 | 1 | 1 | 3 | 3 | 1 | 1 | 3 | 3 | 3 | 3 | 1 | 1 | 3 | 3 |
| | | B | 0 | 0 | 1 | 1 | 3 | 3 | 2 | 1 | 3 | 3 | 3 | 3 | 2 | 2 | 4 | 4 |
| | | C | 0 | 1 | 1 | 1 | 3 | 4 | 2 | 1 | 3 | 3 | 3 | 3 | 2 | 2 | 4 | 4 |
| | | D | 0 | 0 | 1 | 1 | 3 | 3 | 1 | 1 | 3 | 3 | 3 | 4 | 1 | 2 | 4 | 4 |
| | | E | 0 | 0 | 1 | 1 | 3 | 3 | 2 | 1 | 3 | 3 | 3 | 4 | 1 | 1 | 4 | 4 |
| | | F | 0 | 1 | 1 | 1 | 3 | 4 | 1 | 1 | 3 | 3 | 3 | 4 | 1 | 1 | 3 | 4 |
| | K 057 | A | 0 | 0 | 1 | 1 | 0 | 0 | 1 | 1 | 2 | 1 | 3 | 2 | 1 | 1 | 1 | 1 |
| | | B | 0 | 0 | 1 | 1 | 0 | 0 | 1 | 1 | 1 | 1 | 3 | 3 | 2 | 2 | 1 | 1 |
| | | C | 0 | 0 | 1 | 1 | 1 | 1 | 0 | 0 | 0 | 0 | 3 | 3 | 1 | 1 | 1 | 1 |
| | | D | 0 | 0 | 1 | 1 | 0 | 0 | 1 | 0 | 0 | 0 | 2 | 1 | 2 | 2 | 1 | 1 |
| | | E | 0 | 0 | 1 | 1 | 0 | 0 | 1 | 0 | 1 | 1 | 1 | 1 | 2 | 2 | 1 | 1 |
| | | F | 0 | 0 | 1 | 1 | 2 | 2 | 2 | 2 | 2 | 1 | 3 | 1 | 2 | 1 | 1 | 1 |
| | | G | 0 | 0 | 1 | 1 | 1 | 0 | 2 | 2 | 2 | 2 | 1 | 1 | 1 | 1 | 1 | 1 |
| | K 058 | A | 0 | 0 | 2 | 2 | 2 | 2 | 1 | 1 | 0 | 0 | 0 | 0 | 1 | 1 | 2 | 1 |
| | | B | 0 | 0 | 2 | 2 | 2 | 2 | 1 | 1 | 0 | 0 | 1 | 1 | 1 | 1 | 1 | 1 |
| | | C | 1 | 1 | 2 | 2 | 2 | 2 | 1 | 1 | 0 | 0 | 1 | 1 | 1 | 1 | 1 | 1 |
| | | D | 1 | 1 | 2 | 1 | 2 | 2 | 1 | 2 | 0 | 0 | 1 | 1 | 1 | 1 | 1 | 1 |
| | | E | 1 | 1 | 2 | 2 | 2 | 2 | 2 | 2 | 0 | 0 | 0 | 0 | 0 | 0 | 1 | 1 |
| | | F | 0 | 0 | 1 | 1 | 1 | 1 | 2 | 2 | 0 | 1 | 1 | 1 | 2 | 1 | 2 | 2 |
| | | G | 0 | 0 | 1 | 1 | 1 | 1 | 1 | 1 | 1 | 1 | 1 | 2 | 1 | 1 | 2 | 2 |
| | | H | 0 | 1 | 0 | 1 | 1 | 2 | 1 | 2 | 0 | 0 | 1 | 1 | 1 | 0 | 2 | 1 |
| | | I | 0 | 0 | 0 | 1 | 1 | 2 | 1 | 1 | 0 | 0 | 1 | 1 | 1 | 1 | 2 | 1 |
| | | J | 0 | 0 | 0 | 0 | 1 | 1 | 1 | 1 | 0 | 0 | 1 | 1 | 1 | 1 | 2 | 2 |
| | K 059 | A | 0 | 0 | 2 | 2 | 2 | 2 | 2 | 2 | 1 | 0 | 2 | 2 | 2 | 1 | 2 | 2 |
| | | B | 0 | 1 | 2 | 2 | 2 | 2 | 1 | 2 | 0 | 0 | 1 | 2 | 0 | 0 | 1 | 1 |
| | | C | 1 | 1 | 2 | 2 | 1 | 1 | 1 | 1 | 0 | 1 | 2 | 2 | 1 | 1 | 1 | 1 |
| | | D | 1 | 0 | 3 | 3 | 1 | 1 | 2 | 2 | 0 | 0 | 2 | 1 | 1 | 1 | 1 | 1 |
| | | E | 1 | 1 | 1 | 1 | 1 | 1 | 2 | 1 | 3 | 3 | 1 | 1 | 1 | 1 | 1 | 1 |
| | | F | 1 | 1 | 1 | 1 | 1 | 1 | 1 | 2 | 1 | 0 | 2 | 2 | 1 | 1 | 1 | 1 |
| | | G | 1 | 1 | 1 | 1 | 1 | 1 | 2 | 1 | 1 | 1 | 2 | 2 | 2 | 2 | 1 | 1 |
| | | H | 1 | 1 | 1 | 1 | 2 | 2 | 1 | 1 | 1 | 1 | 1 | 2 | 2 | 2 | 2 | 2 |
| | | I | 2 | 2 | 2 | 2 | 2 | 1 | 2 | 2 | 1 | 1 | 1 | 1 | 2 | 1 | 2 | 2 |
| | | J | 0 | 1 | 2 | 2 | 1 | 2 | 2 | 2 | 0 | 0 | 0 | 1 | 2 | 2 | 3 | 2 |
| | K 060 | A | 0 | 0 | 1 | 1 | 3 | 3 | 0 | 1 | 0 | 0 | 2 | 2 | 3 | 2 | 3 | 3 |
| | | B | 1 | 1 | 1 | 2 | 3 | 3 | 0 | 1 | 2 | 2 | 3 | 3 | 2 | 2 | 2 | 3 |
| | | C | 0 | 0 | 1 | 1 | 3 | 3 | 1 | 2 | 1 | 1 | 2 | 3 | 2 | 2 | 2 | 2 |
| | | D | 1 | 1 | 2 | 1 | 3 | 3 | 1 | 1 | 0 | 0 | 2 | 2 | 2 | 3 | 2 | 2 |
| | | E | 0 | 0 | 1 | 1 | 3 | 2 | 1 | 1 | 1 | 1 | 3 | 2 | 2 | 2 | 2 | 2 |
| | | F | 1 | 0 | 2 | 2 | 2 | 2 | 2 | 2 | 2 | 0 | 2 | 2 | 2 | 2 | 2 | 2 |
| | | G | 0 | 1 | 1 | 1 | 2 | 2 | 1 | 1 | 1 | 2 | 2 | 3 | 2 | 2 | 2 | 2 |
| | | H | 0 | 1 | 1 | 2 | 2 | 2 | 2 | 1 | 1 | 1 | 2 | 2 | 2 | 2 | 2 | 2 |
| | | I | 0 | 0 | 1 | 1 | 2 | 2 | 2 | 2 | 1 | 1 | 2 | 2 | 1 | 2 | 1 | 2 |
| | | J | 1 | 1 | 2 | 2 | 2 | 2 | 2 | 1 | 1 | 1 | 2 | 3 | 1 | 1 | 1 | 1 |
| | | K | 0 | 0 | 1 | 1 | 2 | 2 | 0 | 1 | 0 | 0 | 1 | 2 | 0 | 0 | 1 | 1 |
| | | L | 0 | 0 | 1 | 1 | 2 | 2 | 1 | 0 | 0 | 0 | 1 | 2 | 0 | 1 | 1 | 1 |
| | K 063 | A | 0 | 0 | 1 | 1 | 2 | 2 | 2 | 1 | 0 | 0 | 2 | 2 | 1 | 0 | 1 | 1 |
| | | B | 0 | 0 | 1 | 1 | 2 | 2 | 2 | 1 | 0 | 0 | 2 | 2 | 1 | 1 | 1 | 1 |
| | | C | 0 | 0 | 2 | 1 | 2 | 2 | 2 | 2 | 0 | 0 | 2 | 2 | 1 | 1 | 1 | 1 |
| | | D | 1 | 0 | 2 | 2 | 3 | 2 | 2 | 2 | 1 | 1 | 3 | 3 | 1 | 1 | 2 | 2 |
| | | E | 1 | 1 | 2 | 2 | 3 | 2 | 2 | 2 | 1 | 1 | 3 | 3 | 1 | 1 | 2 | 2 |
| | | F | 3 | 2 | 2 | 2 | 3 | 3 | 2 | 2 | 3 | 2 | 2 | 2 | 2 | 2 | 3 | 3 |
| | | G | 2 | 0 | 1 | 1 | 2 | 2 | 2 | 2 | 2 | 1 | 2 | 2 | 3 | 2 | 3 | 2 |
| | | H | 1 | 1 | 1 | 1 | 2 | 2 | 2 | 1 | 0 | 0 | 1 | 1 | 2 | 2 | 2 | 2 |
| | | I | 0 | 0 | 1 | 1 | 2 | 2 | 2 | 1 | 0 | 0 | 0 | 1 | 2 | 2 | 2 | 2 |
| Control | | | | | | | | | | | | | | | | | | |
| | K 054 | A | 1 | 1 | 1 | 1 | 3 | 3 | 4 | 4 | 4 | 4 | 3 | 3 | 4 | 4 | 3 | 4 |
| | | B | 1 | 1 | 2 | 2 | 3 | 3 | 4 | 4 | 4 | 4 | 3 | 3 | 4 | 4 | 3 | 4 |
| | | C | 1 | 0 | 1 | 2 | 3 | 3 | 4 | 4 | 4 | 4 | 3 | 3 | 4 | 4 | 3 | 4 |
| | | D | 0 | 0 | 1 | 2 | 3 | 3 | 4 | 4 | 4 | 4 | 3 | 3 | 4 | 4 | 3 | 4 |
| | | E | 0 | 0 | 2 | 2 | 3 | 3 | 4 | 4 | 4 | 4 | 3 | 3 | 4 | 4 | 3 | 4 |
| | | F | 0 | 0 | 1 | 2 | 3 | 3 | 4 | 4 | 4 | 4 | 3 | 3 | 4 | 4 | 3 | 4 |
| | | G | 0 | 0 | 1 | 2 | 3 | 3 | 4 | 4 | 4 | 4 | 3 | 3 | 4 | 4 | 3 | 4 |
| | K 057 | A | 0 | 0 | 3 | 2 | 2 | 2 | 1 | 1 | 1 | 1 | 2 | 1 | 1 | 1 | 1 | 1 |
| | | B | 0 | 0 | 3 | 2 | 3 | 2 | 2 | 2 | 2 | 2 | 2 | 2 | 1 | 1 | 1 | 1 |
| | | C | 0 | 0 | 2 | 3 | 2 | 2 | 2 | 2 | 3 | 2 | 2 | 2 | 1 | 1 | 2 | 2 |
| | | D | 1 | 1 | 2 | 2 | 2 | 3 | 2 | 2 | 3 | 2 | 2 | 2 | 1 | 1 | 2 | 2 |
| | | E | 1 | 1 | 2 | 2 | 3 | 2 | 3 | 1 | 2 | 2 | 2 | 2 | 1 | 1 | 2 | 2 |
| | | F | 1 | 1 | 2 | 2 | 2 | 2 | 3 | 3 | 2 | 2 | 3 | 2 | 1 | 1 | 2 | 2 |
| | | G | 1 | 0 | 2 | 2 | 3 | 2 | 3 | 3 | 2 | 2 | 2 | 2 | 1 | 1 | 2 | 2 |
| | | H | 1 | 1 | 2 | 2 | 3 | 2 | 3 | 3 | 2 | 1 | 3 | 3 | 2 | 1 | 2 | 2 |
| | | I | 1 | 0 | 3 | 3 | 3 | 3 | 3 | 2 | 2 | 1 | 3 | 3 | 2 | 1 | 2 | 2 |
| | K 058 | A | 2 | 1 | 3 | 3 | 1 | 1 | 4 | 4 | 2 | 1 | 2 | 2 | 2 | 1 | 1 | 1 |
| | | B | 2 | 1 | 2 | 2 | 1 | 1 | 3 | 3 | 2 | 2 | 2 | 2 | 2 | 2 | 2 | 2 |
| | | C | 2 | 2 | 2 | 2 | 1 | 1 | 4 | 4 | 2 | 2 | 1 | 2 | 2 | 2 | 3 | 2 |
| | | D | 2 | 2 | 3 | 3 | 2 | 2 | 2 | 3 | 3 | 2 | 3 | 3 | 1 | 1 | 2 | 2 |
| | | E | 3 | 3 | 3 | 2 | 2 | 2 | 4 | 4 | 2 | 2 | 3 | 2 | 3 | 2 | 3 | 3 |
| | | F | 2 | 2 | 3 | 3 | 3 | 2 | 4 | 4 | 2 | 2 | 3 | 3 | 2 | 2 | 2 | 2 |
| | | G | 2 | 2 | 2 | 2 | 2 | 2 | 4 | 4 | 2 | 2 | 2 | 2 | 2 | 2 | 2 | 2 |
| | | H | 2 | 2 | 2 | 2 | 2 | 2 | 4 | 4 | 2 | 2 | 2 | 2 | 2 | 2 | 3 | 3 |
| | K 059 | A | 0 | 0 | 1 | 2 | 1 | 1 | 2 | 3 | 4 | 4 | 2 | 2 | 1 | 1 | 2 | 2 |
| | | B | 2 | 2 | 1 | 2 | 3 | 2 | 2 | 3 | 4 | 3 | 3 | 3 | 1 | 1 | 3 | 3 |
| | | C | 3 | 2 | 1 | 1 | 2 | 3 | 4 | 4 | 3 | 3 | 3 | 3 | 2 | 2 | 3 | 3 |
| | | D | 3 | 3 | 1 | 1 | 2 | 3 | 4 | 4 | 4 | 4 | 2 | 2 | 2 | 2 | 3 | 3 |
| | | E | 2 | 3 | 1 | 1 | 3 | 3 | 4 | 4 | 4 | 4 | 2 | 2 | 2 | 1 | 3 | 2 |
| | | F | 3 | 2 | 1 | 1 | 3 | 2 | 4 | 4 | 3 | 3 | 3 | 3 | 1 | 2 | 2 | 2 |
| | | G | 3 | 3 | 1 | 1 | 2 | 3 | 4 | 4 | 4 | 4 | 3 | 2 | 2 | 1 | 3 | 3 |
| | K 060 | A | 2 | 1 | 2 | 2 | 2 | 2 | 2 | 2 | 3 | 3 | 3 | 3 | 2 | 2 | 2 | 2 |
| | | B | 1 | 2 | 2 | 2 | 3 | 3 | 2 | 2 | 4 | 3 | 3 | 3 | 2 | 2 | 3 | 2 |
| | | C | 2 | 2 | 2 | 2 | 3 | 3 | 2 | 2 | 4 | 3 | 3 | 3 | 2 | 2 | 2 | 2 |
| | | D | 1 | 1 | 1 | 1 | 3 | 2 | 2 | 2 | 3 | 3 | 3 | 3 | 1 | 2 | 2 | 2 |
| | | E | 2 | 1 | 1 | 1 | 3 | 3 | 2 | 2 | 2 | 3 | 3 | 3 | 3 | 1 | 3 | 3 |
| | | F | 2 | 2 | 1 | 2 | 3 | 2 | 2 | 1 | 2 | 2 | 3 | 2 | 3 | 2 | 3 | 3 |
| | | G | 1 | 1 | 1 | 1 | 3 | 2 | 2 | 2 | 2 | 3 | 3 | 3 | 2 | 3 | 3 | 2 |
| | | H | 1 | 1 | 2 | 1 | 3 | 2 | 2 | 2 | 3 | 3 | 2 | 3 | 2 | 3 | 2 | 3 |
| | | I | 1 | 1 | 1 | 2 | 3 | 3 | 2 | 2 | 2 | 2 | 3 | 2 | 2 | 2 | 2 | 2 |
| | | J | 2 | 2 | 1 | 1 | 2 | 2 | 2 | 2 | 2 | 3 | 3 | 2 | 2 | 3 | 3 | 2 |
| | | K | 1 | 1 | 1 | 1 | 2 | 2 | 2 | 2 | 2 | 3 | 2 | 3 | 2 | 3 | 2 | 3 |
| | | L | 1 | 1 | 1 | 1 | 2 | 2 | 2 | 2 | 2 | 2 | 2 | 2 | 3 | 2 | 2 | 2 |
| | K 063 | A | 1 | 0 | 2 | 2 | 3 | 2 | 2 | 2 | 2 | 1 | 3 | 4 | 2 | 2 | 3 | 2 |
| | | B | 1 | 1 | 3 | 2 | 3 | 3 | 2 | 2 | 3 | 2 | 3 | 4 | 2 | 2 | 3 | 3 |
| | | C | 1 | 1 | 2 | 1 | 2 | 3 | 2 | 3 | 2 | 2 | 3 | 3 | 1 | 1 | 2 | 3 |
| | | D | 1 | 0 | 1 | 1 | 3 | 2 | 2 | 3 | 3 | 3 | 3 | 3 | 2 | 2 | 3 | 3 |
| | | E | 1 | 1 | 1 | 1 | 3 | 3 | 3 | 3 | 2 | 2 | 3 | 3 | 2 | 2 | 2 | 2 |
| | | F | 1 | 1 | 1 | 1 | 3 | 3 | 3 | 3 | 2 | 2 | 3 | 3 | 2 | 2 | 2 | 3 |
| | | G | 1 | 1 | 1 | 1 | 3 | 3 | 2 | 3 | 2 | 2 | 3 | 3 | 4 | 3 | 3 | 2 |
| | | H | 0 | 0 | 2 | 2 | 3 | 3 | 2 | 2 | 4 | 3 | 3 | 3 | 3 | 3 | 2 | 2 |
| | | I | 1 | 1 | 2 | 2 | 3 | 3 | 2 | 2 | 4 | 4 | 3 | 3 | 3 | 2 | 2 | 2 |
| | | J | 1 | 1 | 1 | 1 | 2 | 2 | 2 | 2 | 2 | 2 | 3 | 3 | 3 | 3 | 2 | 1 |
| | | K | 1 | 0 | 2 | 2 | 3 | 3 | 3 | 3 | 1 | 1 | 3 | 3 | 2 | 2 | 1 | 1 |
| | | L | 1 | 1 | 2 | 2 | 3 | 3 | 3 | 3 | 1 | 1 | 3 | 3 | 2 | 2 | 1 | 1 |

The scoring system shown in Table 1 is described by Sellers et al. (J Bone Joint Surg Am 79: 1452-63, 1997). The sections were evaluated blindly by two observers with use of a histological grading scale. The scale was designed to reduce observer bias, to identify subtle changes during repair, and to allow comparisons between standardized studies.

Grading was done with use of a section taken from the middle of the defect. The total score on the grading scale ranges from 0 points (normal cartilage) to 31 points (no repair tissue). Different individual parameters are scored between a minimum of 0 and maximum of 3-5. The modified scale allowed for the evaluation of all relevant aspects of repair of a full-thickness defect of articular cartilage (Table I). Some categories were designed for the evaluation of the entire defect (i.e., category 1, “filling of the defect” relative to the surface of the normal adjacent cartilage, and category 5, “architecture” within the entire defect, not including the margins). One category (e.g., category 7, “New subch bone”) addressed the repair of subchondral bone, with 100 percent replacement signifying complete regeneration of subchondral bone to the level of the original tidemark. There also were categories for the evaluation of the repair of the articular cartilage (e.g., category 3, “matrix staining,” and category 4, “cellular morphology”) and for the evaluation of specific aspects of repair (e.g., category 2, “integration” of repair tissue with surrounding cartilage, and category 6, “architecture” of the surface; and category 8, formation of a “tidemark”).

When the calculation of a percentage was involved (as for the scores in categories 1, 4, 7, and 8), a reticle was used within the eyepiece of the microscope. In category 1, 100 percent filling of the defect meant that new tissue filled the entire area of the defect (nine square millimeters) and extended to the level of the original joint surface. In category 4, the percentage of new cartilage that demonstrated organization of chondrocytes into vertical columns in the radial zone was calculated by dividing the width of the portion of tissue that demonstrated such columns by the total width of the repair tissue (three millimeters). In category 7, the percentage of new subchondral bone was calculated by measuring the area beneath the tidemark that was now occupied by new bone. In category 8, the formation of the tidemark was determined by dividing the width of the portion of the defect that had a new tidemark by the original width of the defect (three millimeters).

The architecture within the defect (category 5) was graded by determining if there were any voids within the repair tissue that were not connected to the surface (with the score dependent on the size and number of voids) or if there were large clefts and fissures associated with a collapsed joint surface.

The total scores as well as the scores for each category were compared among the experimental groups. Statistical analysis of the total scores was performed with the Student t test. score system—derived from Sellers et al (J Bone Joint Surg Am 79: 1452-63, 1997. The statistical analysis of the differences between the rabbits that received the AAV vector encoding FGF-2 and the control group is shown in Table 2.

TABLE 2
Effects of FGF-2-Expression on the histological grading of the repair tissue

| | Control | FGF-2 | | |
|---|---|---|---|---|
| Category | Mean (95% CI) | Mean (95% CI) | F-test* | P value^{†} |
| Filling of defect | 1.22 (0.82-1.64) | 0.40 (0.02-0.78) | 5.75 | <0.05^{†} |
| Integration | 1.73 (1.36-2.10) | 1.27 (0.91-1.64) | 1.95 | 0.08 |
| Matrix staining | 2.45 (1.80-3.11) | 1.88 (1.22-2.53) | 1.40 | 0.19 |
| Cell morphology | 2.98 (2.40-3.57) | 1.34 (0.75-1.92) | 19.49 | <0.001^{†} |
| Architecture of | 2.73 (1.88-3.58) | 1.05 (0.30-1.80) | 9.78 | <0.01^{†} |
| defect | | | | |
| Architecture of | 2.63 (2.07-3.19) | 1.94 (1.38-2.50) | 1.94 | 0.08 |
| surface | | | | |
| Subchondral bone | 2.15 (1.49-2.82) | 1.36 (0.70-2.03) | 1.87 | 0.09 |
| Tidemark | 2.43 (1.71-3.15) | 1.87 (1.15-2.60) | 1.46 | 0.25 |
| Average total score | 18.5 (15.5-21.2) | 11.0 (8.2-14.0) | 15.65 | <0.01^{†} |

*Points for each category and total score were compared between FGF-2 and control groups using a mixed general linear model with repeated-measures analysis of variance (knees nested within the same animals).
^{†}Significant treatment effect.

As indicated in Table 2, statistically significant differences exist between those rabbits that received the AAV vector encoding FGF-2 and the control vector. The Safranin O staining was more intense in the treated knees, i.e. there was an increase in proteoglycan synthesis in knees that received AAV FGF-2. There were also more rounded cells in the FGF-2 treated knees. This cellular morphology is indicative of a chondrocytic phenotype. The new cartilage in the treated samples is also better integrated with the surrounding cartilage than in the control treated samples, a feature also apparent in the anti-collagen II immunohistochemistry.

Knees from rabbits that received AAV-IGF-1R were also sectioned at the four-month time point. Healing in these knees did not progress as it did in knees that received AAV-FGF-2. In fact, at the four-month time point the defects in the rabbits that received AAV-IGF-1R more closely resembled control knees. While cells staining positive for IGF1R using a specific antibody were present, there were fewer of these positive cells than are present in knees that received FGF-2, and fewer cells were present within these defects. The number of transgene-positive cells appears decreased relative to controls that received only AAV-Beta-Gal. These particular animals did not receive the vector encoding the receptor in combination with the vector encoding IGF. It is possible that over-expression of the receptor in the absence of its ligand produced dysfunction.

As described herein, we have shown that AAV expression vectors successfully delivered and expressed therapeutic genes persistently in cells within and surrounding discrete defects introduced in hyaline cartilage in a rabbit model of acute articular cartilage injury. We also showed a therapeutic effect following delivery of the gene cassette encoding FGF-2. FGF-2 expression in articular cartilage improved healing and demonstrated that AAV-mediated delivery of therapeutic gene sequences is useful for the repair of articular cartilage damage in a well-accepted animal model of cartilage disease.

Cartilage Explants

Cartilage explant cultures are employed to explore gene expression efficacy in a complex mixed culture system that retains many of the cell-cell interactions present in native tissue. Articular cartilage explants are prepared from the radiocarpal joints of 1- to 2-week-old calves as 6.2 mm diameter cartilage disks and individually incubated in 96-well plates containing basal medium with 2% FBS. Fresh chondrocytes are then transplanted onto the cartilage discs (0.8×10⁶ cells/disk) after pretreatment with 1 U/ml chondroitin ABC lyase (ICN, Irvine, Calif., USA) in PBS for 1 hour at 37° C.

The chondrocytes are transplanted onto the articular surfaces of cultured cartilage discs after AAV transduction, or AAV can be applied to the disc after the cells have been transplanted (Madry et al., Gene Ther. 19:1443-9, 2001).

Screening of Candidate Therapeutic Vectors in Animal Models.

Rabbit Acute Osteochondral Defect Model

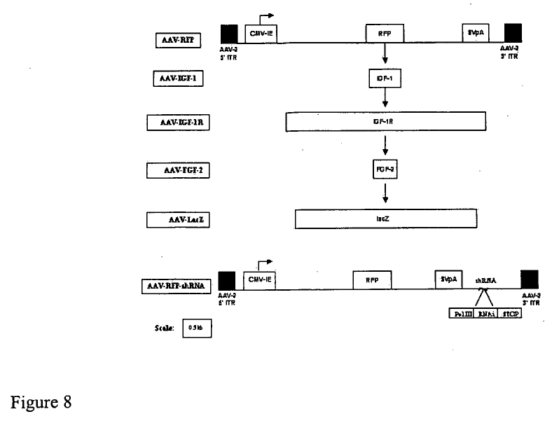

The effects of local overexpression in the knee joint of candidate polypeptides on the repair of full-thickness osteochondral defects, after exposure of cells in and around the defect to AAV cassettes encoding these sequences, is carried out as follows. A schematic diagram of exemplary AAV therapeutic vectors is provided at FIG. 8.

Osteochondral defects are introduced in the femoropatellar groove of adult male Chinchilla Bastard white rabbits as a standard, clinically relevant defect model. Briefly, young male Chinchilla Bastard rabbits (mean weight 3.0 kg) are anesthetized by intramuscular injection. The knee joint is entered through a medial parapatellar approach. The patella is dislocated laterally and the knee flexed to 90°. Two cylindrical osteochondral cartilage defects are created in the patellar groove and the femoral condoyle with a manual cannulated burr (3.2 mm diameter). Each defect is washed with saline and blotted dry. In cases where a single therapeutic vector is applied, 10 ul of AAV is then applied to each defect. Where two vectors will be applied, 10 ul of each will be mixed together and added in two aliquots, with 5 minutes in between applications to encourage adsorption. Each animal receives a marker gene in one knee as a negative control, and the test gene(s) in the other knee.

Studies in a Rabbit Meniscectomy Model of Osteoarthritis

The effect of therapeutic vector expression on osteoarthritis is carried out in a meniscectomy model of osteoarthritis (OA) in rabbits. This model is known in the art (e.g., Lefkoe et al., J. Rheumatol 24:1155-63, 1997; Fernandes et al., Am J Pathol 154: 1159-69, 1999; Messner et al., Osteoarthritis Cartilage 8: 197-206, 2000; Hanashi et al., J. Orthop Sci. 7: 672-6, 2002, each of which is incorporated by reference). In this model a partial meniscectomy of the right knee is performed through a medial parapatellar incision. Because of the more extensive surgery, the procedure is only performed on one knee of each animal. Control animals receive only the AAV-RFP vector, or surgery but no vector. 10-12 animals will be used for each time point.

AAV is delivered by direct intra-articular injection after surgery, rather than at the time of the procedure. This method is expected to be particularly efficacious for vectors that deliver secreted products, and has been used with some success with Adenovirus vectors. Given that AAV is approximately 1/10^{th} the size of Adenovirus, AAV is likely to more easily penetrate into the joint tissue. These experiments are carried out as follows.

AAV is delivered 1 week after surgery. In tissue, AAV transgene expression is typically detectable after several days. Transgene expression levels usually peak for 10 days to two weeks and are generally stable for periods of months to years, as observed in the acute defect model. Differences between treated and untreated knees, with the onset of OA, are expected to be detectable within 8 weeks after surgery. A 12-week time point will be used in the initial study. If differences between groups are seen, in the follow-up series the gene treatments will be applied later, 4-6 weeks after surgery, and the time before collection moved back to 16-18 weeks. Early administration of a successful gene treatment will likely promote healing of the defect which leads to the OA, as our own trials in the defect model indicate. It is therefore possible the gene treatment will remove the underlying cause of the OA instead of, or in addition to, halting its progression. Staggering the time points in this way allows discrimination between these possibilities.

Screening of tissue sections for transgene expression and pathology is carried out as described above, except in this case the sections are evaluated for the prevention or slowing of erosion and degradation, instead of improved pace or quality of healing. The severity of macroscopic and microscopic changes on cartilage on the medial and femoral condyles, and tibial plateaus and synovium, is graded separately and independently by at least 2 individuals. Specifically, significant reductions in the width of osteophytes, in the size of macroscopic lesions, and in the severity of histologic cartilage lesions indicates that a therapeutic vector is useful for the treatment of articular cartilage damage. Therapeutic vectors encoding candidate polypeptides that enhance cartilage repair are identified by comparing the repair of articular cartilage damage in a joint that received the candidate polypeptide relative to a control joint.

In Situ Hybridization in Tissue Sections

In situ hybridization is performed using methods known in the art and described in (Aigner et al., Histopathology 35:373-9, 1999, Gelse et al Osteoarthritis Cartilage 11:141-8, 2003). Deparaffinized and dehydrated sections are digested with proteinase K, post-fixed, washed, acetylated, washed again, and dehydrated. The sections are then hybridized for 12-16 hours at 43° C. After hybridization, the tissue sections are washed at 40° C. in 2×SSC (1×SSC is 0.15M NaCl and 0.15M sodium citrate) and then in 0.5×SSC, treated with RNases A and T1, and washed again for 2 hours at 50° C. with 0.1×SSC. After another wash in 0.5×SSC, the sections are blocked with 3% H₂O₂ for 30 minutes. After a further blocking step in TNB solution (0.1M Tris HCl, pH 7.5; 0.15 NaCl; 0.5% DuPont blocking reagent), sections are incubated with peroxidase labeled streptavidin. Immunodetection is then performed using the TYRAMIDE SIGNAI AMPLIFICATION (TSA) SYSTEM from DuPont (Wilmington, Del.) (TSA indirect system for indirect in situ hybridization ISH).

Statistics

On the basis of literature values for selected cartilage repair procedures, a standard deviation of 25% for the mean total score was estimated to determine the sample size. For a power of 80% and a two-tailed alpha level of 0.05, a sample size of six animals per group would be required to detect a mean difference of 5 points between the groups assuming a pooled standard deviation of 2.5 points (effect size=5/2.5=2.0) using the two-sample Student's t-test. t least 7 animals will be collected from each treatment group at each time point, in the case of the acute injury model, and a minimum of 10 animals for the OA model. For each knee, we examine about 10 sections stained by Safranin O, and analyze it for 8 parameters.

FGF-2 and IGF-1 Effects on Joint Repair

The effects of FGF-2 and IGF-1 on joint repair are likely to exhibit significant complementarity. They act by different mechanisms, and may therefore reinforce each other's effects or act synergistically when delivered over time together. Vectors encoding these two vectors can be applied in combination. Optionally, a therapeutic vector encoding the IGF-1 receptor can also be administered with this combination. By delivering the receptor as well as its ligand, the action of the growth factor is likely to be enhanced by setting up an autocrine loop. The ligand-receptor combination is also likely to be beneficial if down-regulation of the native IGF-1 receptor occurs after prolonged exposure to high levels of IGF-1, as has been reported in experiments using recombinant proteins (Bhaumick et al., Horm Res 35: 246-51, 1991; Geary et al., Horm Metab Res 21: 1-3, 1989).

Animal Models

Although AAV is a human virus, recombinant AAV vectors function efficiently not only in many types of human cells, but also in those of other species, including rats, mice, rabbits, dogs, horses, and primates. This suggests that AAV therapeutic vectors are useful for the treatment of a variety of mammals.

Methods for Delivering Therapeutic Polypeptides to Articular Cartilage

As described herein, AAV vectors are useful for the in vivo delivery of therapeutic molecules to damaged articular cartilage cells in a subject. The stable delivery of therapeutic polypeptides (e.g., FGF-2, IGF-1, and IGF-1R), or fragments thereof, is useful for the repair of damaged cartilage.

Transducing viral (e.g., retroviral, adenoviral, and adeno-associated viral) vectors can be used to express heterologous sequences in somatic cells, because of their high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). Most preferred are AAV vectors, which impose no block to superinfection, and allow the same target populations to be successfully transduced simultaneously with more than one vector. This feature makes it much easier to target cells with more than one transgene, rather than being forced to build every desirable combination into a new vector. This overcomes constraints on the size of the DNA that can be packaged. These features make AAV useful as a research tool and for gene therapy applications. Methods of gene therapy using AAV gain in human gene therapy trials are described in Kay et al., Nat Genet. 24: 257-61, 2000.

While an exemplary AAV-2 vector is described above, any AAV expression vector can be used for in vivo gene delivery (e.g., AAV-1, AAV-2, AAV-3, AAV-4, AAV-5, and AAV-6). AAV expression vectors are known to the skilled artisan and are commercially available from GENEDETECT.COM (Sarasota, Fla.).

A full length gene (e.g., a gene encoding a therapeutic polypeptide), or a portion thereof, can be cloned into a viral vector and expression can be driven from its endogenous promoter or from a promoter specifically expressed in a target cell type of interest (e.g., a cell present in articular cartilage). Other viral vectors that can be used include, for example, a vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; Le Gal La Salle et al., Science 259:988-990, 1993; and Johnson, Chest 107:77 S-83S, 1995). Retroviral vectors are particularly well developed and have been used in clinical settings (Rosenberg et al., N. Engl. J. Med 323:370, 1990; Anderson et al., U.S. Pat. No. 5,399,346).

Non-viral approaches can also be employed for the introduction of therapeutic nucleic acids to a cell of a patient having articular cartilage damage. For example, a nucleic acid molecule can be introduced into a cell by administering the nucleic acid in the presence of lipofection (Felgner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). Preferably the nucleic acids are administered in combination with a liposome and protamine.

Gene transfer can also be achieved using non-viral means involving transfection in vitro. Such methods include the use of calcium phosphate, DEAE dextran, electroporation, and protoplast fusion. Liposomes can also be potentially beneficial for delivery of DNA into a cell. Transplantation of normal genes into the affected tissues of a patient can also be accomplished by transferring a normal nucleic acid into a cultivatable cell type ex vivo (e.g., an autologous or heterologous primary cell or progeny thereof), after which the cell (or its descendants) are injected into a targeted tissue.

cDNA expression for use in gene therapy methods can be directed from any suitable promoter (e.g., any promoter that is expressed in cartilage), and regulated by any appropriate mammalian regulatory element. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

### OTHER EMBODIMENTS

From the foregoing description, it will be apparent that variations and modifications may be made to the invention described herein to adapt it to various usages and conditions. Such embodiments are also within the scope of the following claims.

All publications mentioned in this specification are herein incorporated by reference to the same extent as if each independent publication was specifically and individually indicated to be incorporated by reference.

What is claimed is:

1. A method for enhancing cartilage repair in a subject, said method comprising administering to said subject having cartilage damage at least one vector encoding a therapeutic polypeptide, or fragment thereof, selected from the group consisting of FGF-2, IGF-1, and IGF-1 receptor.

2. The method of claim 1, wherein said vector is an adeno-associated viral vector (AAV) selected from the group consisting of AAV-1, AAV-2, AAV-3, AAV-4, AAV-5, and AAV-6.

3. The method of claim 2, wherein said AAV is AAV-2.

4. The method of claim 3, wherein said therapeutic polypeptide is FGF-2.

5. The method of claim 1, wherein at least two vectors encoding said therapeutic polypeptides are administered.

6. The method of claim 5, wherein one of said vectors encodes an IGF-1 polypeptide and the second vector encodes an IGF-1 receptor.

7. The method of claim 5, wherein one of said vectors encodes an IGF-1 polypeptide and the second vector encodes an FGF-2 polypeptide.

8. The method of claim 1, wherein said cartilage damage results from trauma.

9. The method of claim 1, wherein said cartilage damage results from osteoarthritis.

10. The method of claim 1, wherein said vector is administered to a joint selected from the group consisting of knee, ankle, foot, hip, spine, wrist, elbow, and shoulder.

11. An AAV vector comprising an open reading frame that encodes an IGF-1 or IGF-1 receptor polypeptide, or a fragment thereof.

12. The vector of claim 11, wherein said vector further comprises an open reading frame that encodes an FGF-2 polypeptide.

13. The vector of claim 12, wherein said vector further comprises a promoter operably linked to said nucleic acid molecule, and capable of driving the expression of said nucleic acid molecule in a specific cell type, tissue, or organ.

14. A cell comprising the vector of claim 11.

15. A pharmaceutical composition comprising an AAV vector that encodes an IGF-1 polypeptide or an IGF-1 receptor polypeptide and an excipient.

16. A cartilaginous cell comprising an AAV vector that encodes FGF-2, or a fragment thereof.

17. A method for identifying a candidate polypeptide that enhances cartilage repair, said method comprising:
(a) contacting an organism having cartilage damage with at least one AAV vector that encodes a candidate polypeptide; and
(b) detecting cartilage repair in said organism relative to a control organism not contacted with said vector, wherein said repair indicates that said candidate polypeptide enhances cartilage repair.

18. The method of claim 17, wherein said vector is an AAV vector selected from the group consisting of AAV-1, AAV-2, AAV-3, AAV-4, AAV-5, or AAV-6.

19. The method of claim 17, wherein said polypeptide is a growth factor or growth factor receptor polypeptide.

20. The method of claim 17, wherein said vector is administered directly to an articular joint.

21. A kit comprising an AAV vector that encodes FGF-2, IGF-1, or an IGF-1 receptor and instructions for administering at least one of said vectors to a subject having articular cartilage damage.
